(19) United States
(12) Patent Application Publication
(10) Pub. No.: US 2009/0325933 A1
(43) Pub. Date: Dec. 31, 2009

(54) Pyrimidinone Derivatives and Their Use as a Drug

(75) Inventors: Lydie Poitout, Antony (FR); Carole Sackur, Paris (FR); Eric Ferrandis, Saint Remy Les Chevreuse (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Ipsen Pharma S.A.S., Boulogne-Billancourt (FR)

(21) Appl. No.: 12/375,212

(22) PCT Filed: Jul. 13, 2007
(86) PCT No.: PCT/FR07/01205

§ 371 Date: Mar. 11, 2009

(30) Foreign Application Priority Data
Jul. 27, 2006 (FR) 0606864

(51) Int. Cl. A61K 31/519; C07D 495/04; A61K 31/5377; A61K 31/55; A61P 35/00; A61P 29/00; A61P 37/00; A61P 19/00; A61P 25/00

(52) U.S. Cl. 514/217.06; 544/278; 544/117; 540/600; 514/260.1; 514/234.2

(57) ABSTRACT

The present application relates to new pyrimidinone derivatives. These products have a good affinity for certain sub-types of cannabinoid receptors, especially the CB2 receptors. They are particularly attractive for treating pathological conditions and diseases in which one or more cannabinoid receptors are involved. The invention also relates to pharmaceutical compositions containing said products and their use for the preparation of a drug.

A subject of the present application is novel pyrimidinone derivatives. These products have a good affinity for certain sub-types of cannabinoid receptors, in particular the CB2 receptors. They are particularly useful for treating pathological states and diseases in which one or more cannabinoid receptors are involved. The invention also relates to pharmaceutical compositions containing said products and their use for the preparation of a medicament.

The cannabinoids are psychoactive components present in Indian cannabis (Cannabis sativa) including approximately 6 different molecules, the most represented of which is delta-9-tetrahydrocannabinol. Knowledge of the therapeutic activity of cannabis goes back to the ancient Chinese dynasties in which, 5,000 years ago, cannabis was used for the treatment of asthma, migraines and gynaecological disorders. It was in 1850 that cannabis extracts were recognized and included in the American pharmacopeia.

The cannabinoids are known for having different effects on numerous functions and organs, the most important being on the central nervous system and on the cardiovascular system. These effects include alterations to the memory, euphoria and sedation. The cannabinoids also increase the pulse and modify the systemic arterial pressure. Peripheral effects linked to bronchial constriction, immunomodulation and inflammation have also been observed. More recently, it has been shown that the cannabinoids suppress the cellular and humoral immune responses and possess anti-inflammatory properties. Despite all of these properties, the therapeutic use of the cannabinoids is controversial because of their psychoactive effects (cause of dependency) but also for their multiple side effects which have not yet been completely characterized. Although numerous works have been carried out in this field since the 1940s, little information exists on the characterization of cannabinoid receptors, the existence of endogenous ligands and until recently on selective products of a particular receptor sub-type.

Two cannabinoid receptors have been identified and cloned, CB1 and CB2. CB1 is expressed predominantly in the central nervous system whereas CB2 is expressed in the peripheral tissues, mainly in the immune system. These 2 receptors are members of the family of the receptors coupled to the G proteins and their inhibition is linked to the activity of adenylate cyclase.

On the basis of all this information, a need exists for compounds capable of selectively modulating the cannabinoid receptor and therefore the pathologies associated with such receptors. Thus, CB2 modulators offer a single pharmacotherapeutical approach against immune disorders, inflammation, osteoporosis, renal ischaemia and other pathological states. There is considerable interest in developing cannabinoid analogues possessing a strong affinity for the CB2 receptor. Cannabinoid analogues which specifically modulate the CB2 receptor, directly or indirectly, can produce clinically useful effects without affecting the central nervous system, thus providing a rational therapeutic approach for a wide variety of pathological states.

The novel compounds of this invention modulate the activity of CB2 and are therefore as a result useful for the treatment and the prevention of the pathological states and diseases associated with the activity of the cannabinoid receptors such as, but not limited to, cell proliferation disorders such as cancer, immune disorders, inflammation, pain, osteoporosis, epilepsy, nausea associated with chemotherapy treatments, fibrosis, gastro-intestinal disorders, neurodegenerative diseases including multiple sclerosis and dyskinesia, Parkinson's disease, Huntington's chorea, Alzheimer's disease but also for preventing or curing diseases associated with motor function such as Tourette's syndrome, and providing neuroprotection.

A subject of the invention is therefore compounds of general formula (I)

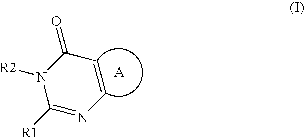

in racemic, or enantiomeric form or any combinations of these forms and in which:
R₁ represents a radical corresponding to the anthracene group, a —Y₁—V₁-Z₁ radical or of formula

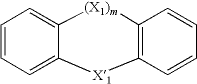

X₁ and X′₁ represent, independently, —CH₂—, —C(O)—, —O—, —S— or —NH—;
m represents 0 or 1;
Y₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
V₁ represents a covalent bond, —O—, —S—, —NH—, —C(O)— or (C₁-C₂)alkyl;
Z₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
R₂ represents a radical of formula-(CH₂)₂—R′₂;
R′₂ represents a (C₃-C₇)cycloalkyl, bicycloalkyl, heterocycloalkyl, heterobicycloalkyl, cyclohexenyl, aryl or heteroaryl radical, all these radicals being optionally substituted by one or more identical or different substituents chosen from: halo, (C₁-C₆)alkyl, (C₁-C₆)haloalkyl, (C₁-C₆)alkoxy and (C₁-C₆)haloalkoxy;
A represents a condensed mono- or bi-cyclic, unsaturated, aromatic or non-aromatic radical, containing a heteroatom chosen from O and S and optionally substituted by one or more identical or different radicals, chosen from: halo, nitro, (C₁-C₆)alkyl, (C₁-C₆)haloalkyl, (C₁-C₆)alkoxy, (C₁-C₆)haloalkoxy and aryl optionally substituted by one or more substituents chosen from: halo and (C₁-C₆)alkyl;
or a pharmaceutically acceptable salt thereof,
excluding the compounds of formula (Ic)

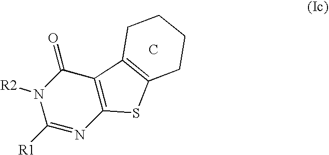

in which the ring C is an unsaturated carbon-containing ring containing at the most 3 double bonds and optionally substituted.

In the definitions indicated above, the expression halo represents the fluoro, chloro, bromo or iodo radical, preferably chloro, fluoro or bromo. The expression (C₁-C₆)alkyl (unless otherwise specified), preferably represents an alkyl radical having 1 to 6 carbon atoms, linear or branched, such as the methyl, ethyl, propyl, isopropyl, butyl, isobutyl, sec-butyl and tert-butyl, pentyl or amyl, isopentyl, neopentyl, hexyl or isohexyl radicals. The expression (C₁-C₂)alkyl represents an alkyl radical having 1 to 2 carbon atoms as defined above.

By haloalkyl is meant an alkyl radical at least one of the hydrogen atoms (and to optionally all) of which is replaced by a halogen atom such as for example trifluoromethyl.

The term alkoxy denotes the radicals in which the alkyl radical is as defined above such as for example the methoxy, ethoxy, propyloxy or isopropyloxy radicals but also secondary or tertiary linear butoxy, pentyloxy. By haloalkoxy is meant an alkoxy radical at least one of the hydrogen atoms (and optionally all) of which is replaced by a halogen atom such as for example trifluoromethoxy, difluoromethoxy, trifluoroethoxy.

The term (C₃-C₇)cycloalkyl denotes a saturated monocyclic carbon-containing system comprising 3 to 7 carbon atoms, namely the cyclopropyl, cyclobutyl, cyclopentyl, cyclohexyl or cycloheptyl rings. The expression heterocycloalkyl denotes a condensed saturated monocyclic or bicyclic system containing 2 to 9 carbon atoms and at least one heteroatom. This radical can contain several identical or different heteroatoms. Preferably, the heteroatoms are chosen from oxygen, sulphur or nitrogen. As examples of heterocycloalkyl, the following rings can be mentioned: pyrrolidine, imidazolidine, pyrrazolidine, isothiazolidine, thiazolidine, isoxazolidine, oxazolidine, piperidine, piperazine, morpholine, azepane (azacycloheptane), azacyclooctane, decahydroisoquinoline (or decahydroquinoline), tetrahydrofuran (tetrahydrofuryl radical), tetrahydropyran, dioxane, dioxolane or tetrahydrothiophene (tetrahydrothienyl radical).

The term bicycloalkyl denotes a non-condensed saturated bicyclic hydrocarbonated system containing 7 to 8 carbon atoms. As examples of bicycloalkyl, there can be mentioned the bicycloheptanes and bicyclooctanes such as bicyclo[2,2,1]heptane, bicyclo[2,2,2]octane or bicyclo[3,2,1]octane.

The term heterobicycloalkyl denotes a non-condensed saturated bicyclic hydrocarbonated system containing 6 to 7 carbon atoms and at least one heteroatom chosen from nitrogen, oxygen and sulphur. As examples of heterobicycloalkyl, there can be mentioned the aza-bicycloheptanes and aza-bicyclooctanes such as 7-aza-bicyclo[2,2,1]heptane, 2-aza-bicyclo[2,2,2]octane or 6-aza-bicyclo[3,2,1]octane.

The expression aryl represents an aromatic radical constituted by a condensed ring or rings, such as for example the phenyl, naphthyl, fluorenyl or anthryl radical.

The expression heteroaryl denotes an aromatic radical, constituted by a condensed ring or rings, with at least one ring containing one or more identical or different heteroatoms chosen from sulphur, nitrogen or oxygen. As examples of heteroaryl radicals, the following radicals can be mentioned: pyrrolyl, imidazolyl, pyrazolyl, isothiazolyl, thiazolyl, isoxazolyl, oxazolyl, oxadiazolyl, triazolyl, thiadiazolyl, pyridyl, pyrazinyl, pyrimidyl, pyridazinyl, quinolyl, isoquinolyl, quinoxalinyl, indolyl, benzotriazolyl, benzothiazolyl, benzoxadiazoyl, carbazolyl, phenoxazinyl, thieno-pyridinyl (thieno[2,3-b]pyridine, thieno[3,2-b]pyridine, thieno[2,3-c]pyridine, thieno[3,2-c]pyridine, thieno[3,4-b]pyridine, thieno[3,4-c]pyridine), thieno-pyrazinyl (thieno[2,3-b]pyrazine, thieno[3,4-b]pyrazine), thienyl, benzothienyl, furyl, benzofuryl, dihydrobenzofuryl, thioxanthenyl, pyranyl, benzopyranyl, dibenzopyrazinyl, acridinyl.

The expression aromatic, unsaturated, condensed, mono- or bi-cyclic radical can be illustrated by the heteroaryl radical as defined above and containing a heteroatom chosen from O or S.

The expression non-aromatic unsaturated, condensed, mono- or bi-cyclic radical and containing at least one heteroatom chosen from O or S, can be illustrated by the heteroaryl radicals as defined above and in which at least one double bond is hydrogenated. There can thus be mentioned as examples the radicals associated with the following rings: dihydrothiophene (2,5-dihydrothiophene, 2,3-dihydrothiophene), tetrahydrobenzothiophene (4,5,6,7-tetrahydro-1-benzothiophene), dihydrocyclopenta-thiophene (5,6-dihydro-4H-cyclopenta[b]thiophene), dihydrofuran, dihydropyrane, tetrahydropyran, dihydrobenzofuran, benzodioxole, dihydro-benzodioxine.

In the compounds of formula (Ic) as defined above, the ring C is a carbon-containing ring constituted only by carbon and hydrogen, containing 1 to 3 double bonds; it is optionally substituted.

A more particular subject of the present invention is a compound of formula I as defined above, characterized in that
R₁ represents a radical corresponding to the anthracene group, a radical of formula —Y₁—V₁-Z₁ or of formula

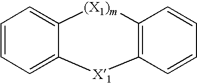

X₁ and X′₁ represent, independently, —CH₂—, —C(O)— or —NH—;
m represents 0 or 1;
Y₁ represents a (C₃-C₇)cycloalkyl, or aryl radical optionally substituted by one or more identical or different halo substituents;
V₁ represents a covalent bond, —O—, —C(O)— or —CH₂—;
Z₁ represents a (C₃-C₇)cycloalkyl, aryl or heteroaryl radical, all these radicals being optionally substituted by one or more identical or different substituents chosen from: halo, (C₁-C₆)alkyl, (C₁-C₆)haloalkyl and (C₁-C₆)alkoxy;
   and preferably
Y₁ represents a cyclohexyl or phenyl radical optionally substituted by one or more identical or different halo substituents;
Z₁ represents a cyclohexyl, phenyl or thienyl radical, all these radicals being optionally substituted by one or more identical or different substituents chosen from: halo, (C₁-C₆)alkyl, (C₁-C₆)haloalkyl and (C₁-C₆)alkoxy.

A more particular subject of the present invention is also a compound of formula I as defined above, characterized in that
R′₂ represents a (C₃-C₇)cycloalkyl, bicycloalkyl, heterocycloalkyl, cyclohexenyl, aryl or heteroaryl radical, all these radicals being optionally substituted by one or more identical or different substituents chosen from: halo, (C₁-C₆)alkyl, (C₁-C₆)haloalkyl, (C₁-C₆)alkoxy;
   and preferably
the (C₃-C₇)cycloalkyl, bicycloalkyl, heterocycloalkyl, aryl or heteroaryl radical represented by R′₂ is chosen from: cyclopentyl, cyclohexyl, cycloheptyl, bicyclo[2,2,1]heptyl, phenyl, pyrrolidinyl, piperidinyl, azepanyl, morpholinyl, tetrahydropyranyl, pyridinyl and thienyl.

A more particular subject of the present invention is also a compound of formula I as defined above, characterized in that A represents an aromatic radical optionally substituted by one or more identical or different substituents chosen from (C₁-C₆)alkyl and aryl. Preferably, A represents the thienyl, furyl or benzothienyl radical, said radicals being optionally substituted by one or more identical or different substituents chosen from (C₁-C₆)alkyl and phenyl.

Preferably, a subject of the present invention is a compound of formula I as defined above, characterized in that
R₁ represents a radical of formula —Y₁—V₁-Z₁;
Y₁ represents a cyclohexyl or phenyl radical;
V₁ represents a covalent bond;
Z₁ represents a cyclohexyl or phenyl radical optionally substituted by one or more identical or different halo, (C₁-C₆)alkyl, (C₁-C₆)haloalkyl substituents.

Preferably, a subject of the present invention is also a compound of formula I as defined above, characterized in that R′₂ represents the piperidinyl, azepanyl, morpholinyl, tetrahydropyranyl, cyclohexyl or cyclohexenyl radical.

Preferably, a subject of the present invention is also a compound of formula I as defined above, characterized in that A represents a monocyclic radical. Very preferably, A represents the thienyl radical optionally substituted by one or more identical or different (C₁-C₆)alkyl substituents.

Preferably, a subject of the present invention is also a compound of general formula (I) as defined above, characterized in that A represents a bicyclic radical. Very preferably, A forms with the pyrimidinone ring, the following compound

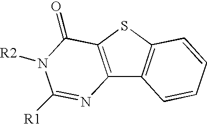

In the present application, the symbol -> * corresponds to the attachment point of the radical. When the attachment site is not specified on the radical, this means that the attachment is carried out on one of the sites of this radical available for such attachment.

According to the definitions of the variable A, R₁ and R₂ groups, the compounds according to the invention can be prepared according to the procedure described below:

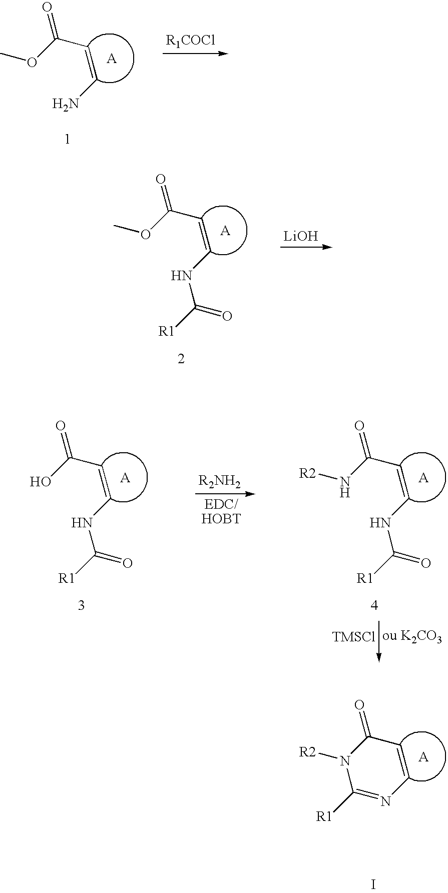

As described in the above diagram, the α-amino ester derivative (1) can be coupled to an acid chloride [commercial or prepared from the corresponding carboxylic acid either by treatment with oxalyl chloride in an aprotic solvent at a temperature of 20 to 80° C., or by treatment with thionyl chloride in the presence or absence of toluene at a temperature of 80 to 110° C., in the presence or absence of dimethylformamide, or by treatment with an equimolar solution of thionyl chloride and benzotriazole in an inert solvent such as methylene chloride, at ambient temperature for 5 minutes to 30 minutes, according to the procedure described by S. S. Chaudhari et al, Synlett, 1999, (11), 1763-1765], in the presence of a tertiary base such as triethylamine or diisopropylethyl diamine, in an inert organic solvent such as methylene chloride, tetrahydrofuran or dimethylformamide at ambient temperature for 3 to 24 hours in order to produce the corresponding amide (2).

The methyl ester (2) can then be saponified in the presence of an inorganic base such as dihydrated lithium hydroxide in a mixture of polar solvents such as water and tetrahydrofuran at a temperature of 20 to 80° C. for 3 to 17 hours or alternatively in a micro-wave oven at a temperature of 100° C. for 15 to 30 minutes. The resultant carboxylic acid (3) can be coupled to a primary amine in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt) in an inert organic solvent such as methylene chloride, tetrahydrofuran or dimethylformamide at ambient temperature for 3 to 24 hours or alternatively heated under micro-waves at a temperature of 80 to 120° C. (Biotage® equipment), in a sealed tube, for 10 to 30 minutes, in order to produce the corresponding diamide (4). The cyclization of the diamide (4) to pyrimidinone (I) can be carried out in the presence of chlorotrimethylsilane (TMSCl) in the presence of a tertiary base, such as triethylamine or N,N-dimethylethylamine in an inert organic solvent such as tetrahydrofuran or acetonitrile at ambient temperature for 3 to 96 hours. Alternatively, the diamide (4) can be treated by an inorganic base such as potassium or caesium carbonate, in the presence or absence of a phase transfer agent such as tetrabutylammonium bromide (TBAB) in an organic solvent such as DMF, at a temperature of 200 to 250° C. under micro-waves (Biotage® equipment), in a sealed tube, for 15 minutes to 2 hours.

### EXAMPLE A

### 2-biphenyl-4-yl-3-(2-piperidin-1-ylethyl)thieno[3,2-d]pyrimidin-4(3H)-one hydrochloride

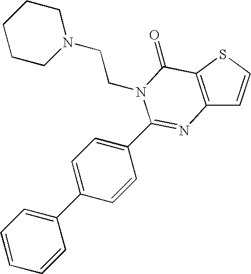

### Stage 1: methyl 3-[(biphenyl-4-ylcarbonyl)amino]thiophene-2-carboxylate

Triethylamine (1.5 eq) and biphenyl-4-carbonyl chloride (3.34 g; 1.2 eq) are successively added to a solution of methyl 3-aminothiophene-2-carboxylate (2 g) in anhydrous dichloromethane (40 ml). The mixture is stirred at ambient temperature for 2 hours then hydrolyzed with 30 ml of water and dichloromethane (40 ml) is added. After decantation and extractions, the combined organic phases are washed with salt water, dried over Na₂SO₄ then concentrated under reduced pressure at 40° C. The solid obtained is washed with a minimum amount of ethyl ether and dried (2.5 g) and used without subsequent purification in the following stage.

NMR (¹H, 400 MHz, DMSO-d₆): δ 3.90 (s, 3H), 7.41-8.24 (m, 12H), 11.05 (s, 1H), 13.6

### Stage 2: 3-[(biphenyl-4-ylcarbonyl)amino]thiophene-2-carboxylic acid

A solution of LiOH.H₂O (1.5 g; 6 eq) in water (20 ml) is added to a solution of methyl 3-[(biphenyl-4-ylcarbonyl)amino]thiophene-2-carboxylate (2 g) in THF (60 ml). The mixture is heated under reflux for 3 hours then cooled down to ambient temperature and concentrated under reduced pressure at 40° C. Ethyl acetate (100 ml) and water (30 ml) are added to the residue. The mixture is acidified by adding acetic acid up to pH 5. After decantation and extractions, the combined organic phases are dried over sodium sulphate and concentrated under reduced pressure at 40° C. The residue is taken up several times with a methanol/toluene mixture and concentrated under reduced pressure in order to eliminate the residual acetic acid. The solid obtained is washed with methanol (1.53 g; 38% from Stage 1).

MS/LC: calculated MM=337.4; m/z=338.2 (MH+)

NMR (¹H, 400 MHz, DMSO-d₆): δ 7.41-8.13 (m, 12H), 11.35 (s, 1H), 13.6 (s, 1H).

### Stage 3: 3-[(biphenyl-4-ylcarbonyl)amino]-N-(2-piperidin-1-ylethyl)thiophene-2-carboxamide

(2-piperidin-1-ylethyl)amine (179 mg; 1.5 eq), HOBT (138 mg; 1.1 eq) in solution in anhydrous THF (2 ml) then EDC.HCl (197 mg; 1.1 eq) in solution in chloroform (3 ml) are successively added to a solution of 3-[(biphenyl-4-ylcarbonyl)amino]thiophene-2-carboxylic acid (300 mg) in anhydrous THF (7 ml) and placed in a “Biotage®” reaction tube. The tube is sealed with a cap, placed in the “Biotage®” micro-wave and heated under magnetic stirring at 100° C. for 20 minutes. The mixture is concentrated under reduced pressure at 40° C. Dichloromethane (30 ml) and water (10 ml) are added to the residue. After decantation and extractions, the combined organic phases are washed with an aqueous solution saturated with hydrogen carbonate then salt water, dried over Na₂SO₄ then concentrated under reduced pressure at 40° C. The 3-[(biphenyl-4-ylcarbonyl)amino]-N-(2-piperidin-1-ylethyl)thiophene-2-carboxamide thus obtained is used in the following stage without purification.

### Stage 4: 2-biphenyl-4-yl-3-(2-piperidin-1-ylethyl)thieno[3,2-d]pyrimidin-4(3H)-one hydrochloride

Potassium carbonate (645 mg) and TBAB (30 mg) are added to a solution of 3-[(biphenyl-4-ylcarbonyl)amino]-N-(2-piperidin-1-ylethyl)thiophene-2-carboxamide in DMF (10 ml) and placed in a “Biotage®” reaction tube. The tube is sealed with a cap, placed in “Biotage®” micro-wave and heated under magnetic stirring at 250° C. for 20 minutes. The mixture is concentrated under reduced pressure at 40° C. Purification by flash chromatography on silica gel (eluant: 100% heptane to 100% ethyl acetate) produces the expected compound in the form of a free base. The corresponding hydrochloride salt is formed by adding a 1N HCl solution in ethyl ether to the solution of the free base in ethyl acetate. The precipitate obtained is filtered and dried in order to produce the expected hydrochloride compound (126 mg, 30% from Stage 3).

MS/LC: calculated MM=415.5; m/z=416.2 (MH+)

NMR (¹H, 400 MHz, DMSO-d₆): δ 1.33 (m, 1H), 1.51-1.78 (m, 5H), 2.81 (m, 2H), 3.28 (m, 4H), 4.32 (t, 2H), 7.45 (m, 2H), 7.53 (m, 2H), 7.76 (m, 4H), 7.88 (m, 2H), 8.28 (d, 1H), 9.33 (s, 1H).

### EXAMPLE B

### 3-(2-cyclohexylethyl)-2-(4-cyclohexylphenyl)thieno[3,2-d]pyrimidin-4(3H)-one

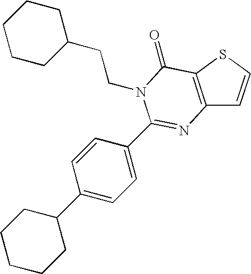

### Stage 1: methyl 3-[(4-cyclohexylbenzoyl)amino]thiophene-2-carboxylate

Preparation of 4-cyclohexylbenzoyl chloride: Benzotriazole (4.47 g) is added to a solution of thionyl chloride (2.73 ml) in anhydrous DCM (25 ml). 20 ml of this solution (1.5 M) are added to a 4-cyclohexylbenzoic acid solution (5 g). The mixture is stirred at ambient temperature for 20 minutes then the benzotriazole chloride precipitate is filtered. MgSO₄.7H₂O (200 mg) is added to the filtrate in order to destroy the residual reagent excess. The mixture is filtered and the filtrate is evaporated under reduced pressure.

The 4-cyclohexylbenzoyl chloride thus obtained is dissolved in anhydrous DCM (10 ml) and added dropwise to a solution of methyl 3-aminothiophene-2-carboxylate (2.7 g) and triethylamine (2.6 ml) in anhydrous DCM (50 ml). The mixture is stirred for 3 hours at ambient temperature then hydrolyzed with 30 ml of water. After decantation and extractions, the combined organic phases are washed with salt water, dried over Na₂SO₄ then concentrated under reduced pressure at 40° C. Purification by flash chromatography on silica gel (eluant: 100% heptane to heptane/ethyl acetate 8:2) produces the expected compound in the form of a white solid (2 g, 34% yield).

NMR (¹H, 400 MHz, DMSO-d₆): δ 1.25 (m, 1H), 1.30-1.52 (m, 4H), 1.71 (d, 1H), 1.80 (m, 4H), 2.60 (t, 1H), 3.88 (s, 3H), 7.45 (AB, 2H), 7.85 (AB, 2H), 7.98 (AB, 1H), 8.11 (AB, 1H), 10.96 (s, 1H).

### Stage 2: 3-[(4-cyclohexylbenzoyl)amino]thiophene-2-carboxylic acid

A solution of LiOH.H₂O (0.75 g; 6 eq) in water (10 ml) is added to a solution of methyl 3-[(4-cyclohexylbenzoyl)amino]thiophene-2-carboxylate (1 g) in THF (30 ml). The mixture is heated under reflux for 3 hours then cooled down to ambient temperature and concentrated under reduced pressure at 40° C. Ethyl acetate (80 ml) and water (20 ml) are added to the residue. The mixture is acidified by adding acetic acid up to pH 5. After decantation and extractions, the combined organic phases are dried over sodium sulphate and concentrated under reduced pressure at 40° C. The residue is taken up several times in a methanol/toluene mixture and concentrated under reduced pressure in order to eliminate the residual acetic acid. The solid obtained is dried (0.95 g; 99%).

NMR (¹H, 400 MHz, DMSO-d₆): δ 1.22 (m, 1H), 1.30-1.52 (m, 4H), 1.71 (d, 1H), 1.80 (m, 4H), 2.60 (t, 1H), 3.88 (s, 3H), 7.43 (AB, 2H), 7.85 (AB, 2H), 7.88 (AB, 1H), 8.10 (AB, 1H), 10.96 (s, 1H), 13.3 (broad s, 1H).

### Stage 3: 3-(2-cyclohexylethyl)-2-(4-cyclohexylphenyl)thieno[3,2-d]pyrimidin-4(3H)-one

(2-cyclohexylethyl)amine (127 mg; 1.5 eq), HOBT (23 mg; 1.1 eq) in solution in anhydrous THF (1 ml) then EDC.HCl (32 mg; 1.1 eq) in solution in chloroform (1 ml) are successively added to a solution of 3-[(biphenyl-4-ylcarbonyl)amino]thiophene-2-carboxylic acid (50 mg) in anhydrous THF (1 ml) and placed in a “Biotage®” reaction tube. The tube is sealed with a cap, placed in “Biotage®” micro-wave and heated under magnetic stirring at 100° C. for 10 minutes. The mixture is concentrated under reduced pressure at 40° C. Dichloromethane (30 ml) and water (10 ml) are added to the residue. After decantation and extractions, the combined organic phases are washed with an aqueous solution saturated with hydrogen carbonate then salt water, dried over Na₂SO₄ then concentrated under reduced pressure at 40° C.

1 ml of a 1.25 M solution of triethylamine in acetonitrile and 1 ml of a 1M solution of trimethylchlorosilane in acetonitrile are added to the 3-[(4-cyclohexylbenzoyl)amino]-N-[2-(tetrahydro-2H-pyran-4-yl)ethyl]thiophene-2-carboxamide thus obtained. The mixture is stirred for 18 hours at ambient temperature then concentrated under reduced pressure at 40° C. Dichloromethane (25 ml) and water (10 ml) are added to the residue. After decantation and extractions, the combined organic phases are washed with an aqueous solution saturated with hydrogen carbonate then salt water, dried over Na₂SO₄ then concentrated under reduced pressure at 40° C. Purification by flash chromatography on silica gel (eluant: heptane 100% to heptane/ethyl acetate 1:1) produces the expected compound (30 mg, 46% yield).

MS/LC: calculated MM=420.6; m/z=421.3 (MH+)

NMR (¹H, 400 MHz, DMSO-d₆): δ 0.61 (m, 2H), 1.01 (m, 4H), 1.22-1.82 (m, 17H), 2.60 (t, 1H), 3.93 (t, 2H), 7.37 (m, 3H), 7.50 (AB, 2H), 8.19 (AB, 1H).

In a manner analogous to the procedure described for 2-biphenyl-4-yl-3-(2-piperidin-1-ylethyl)thieno[3,2-d]pyrimidin-4(3H)-one and 3-(2-cyclohexylethyl)-2-(4-cyclohexylphenyl)thieno[3,2-d]pyrimidin-4(3H)-one, the following compounds were prepared:

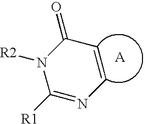

in which R₁ represents one of the radicals below:

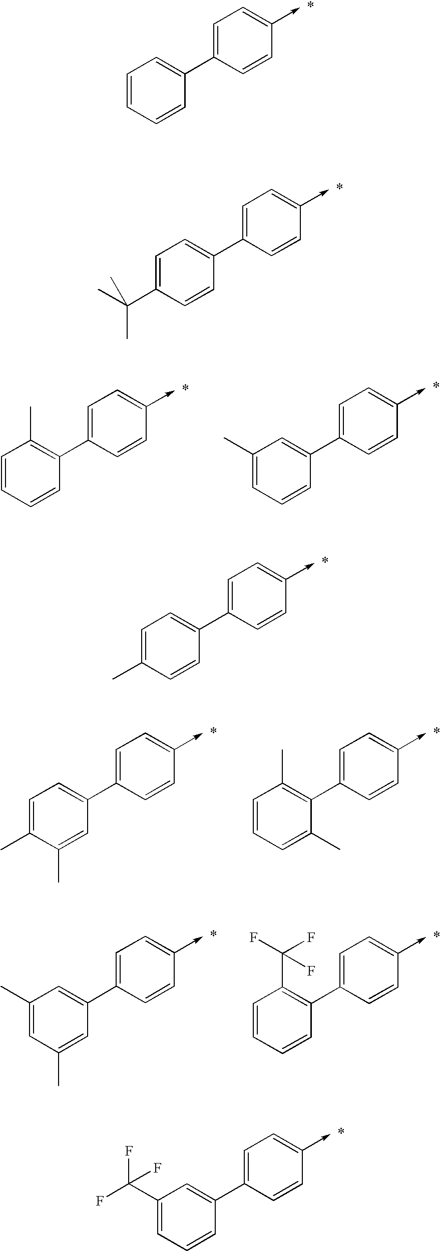

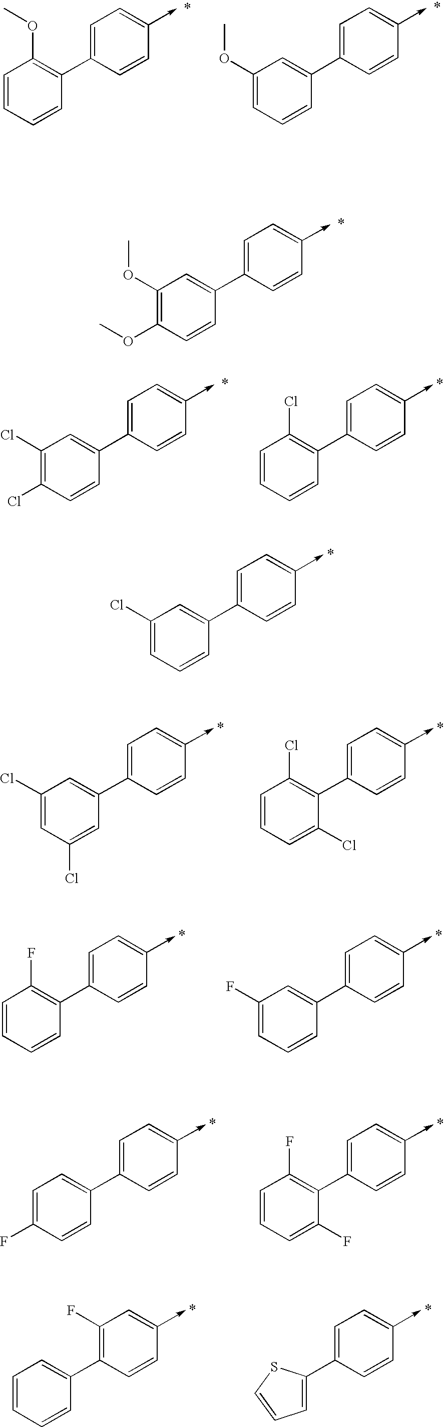

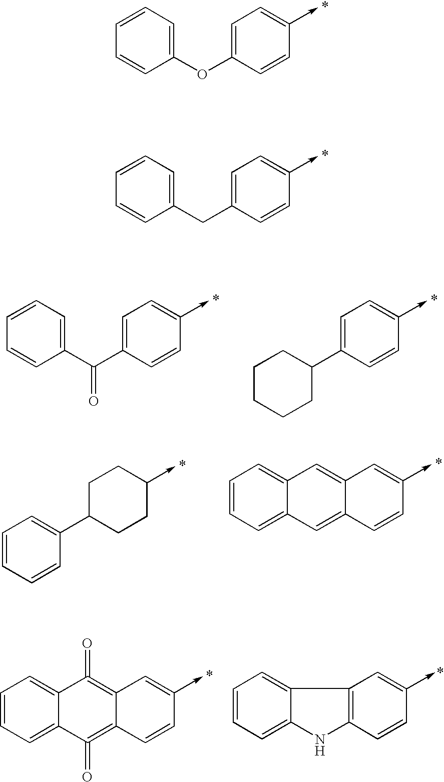

R₂ represents one of the radicals below:

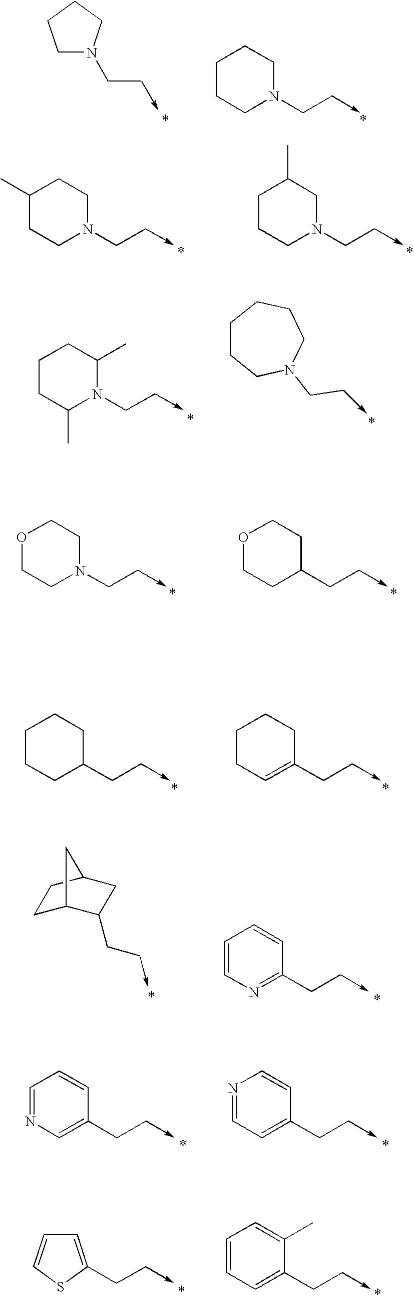

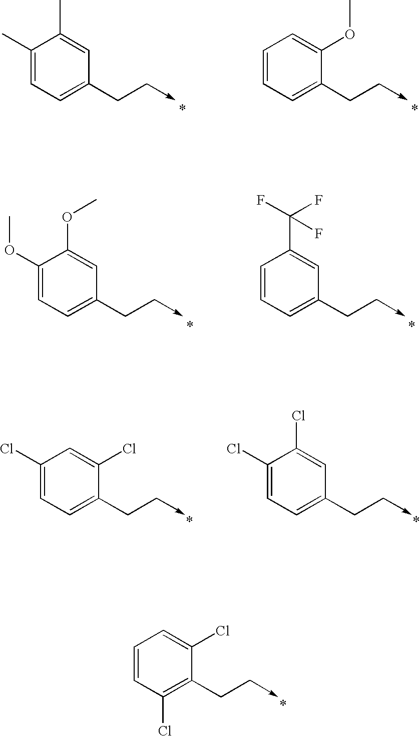

and

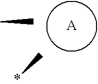

represents one of the radicals below:

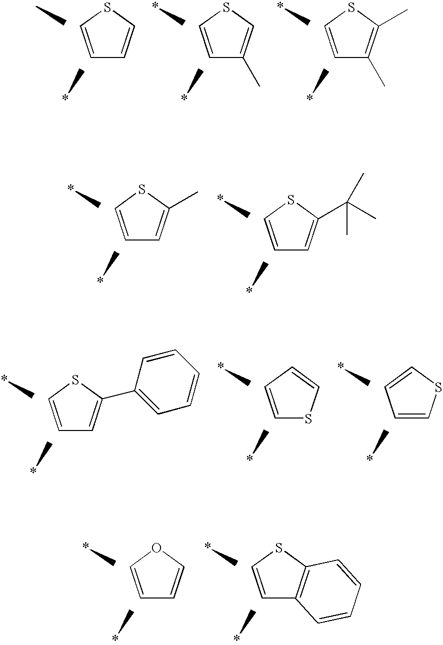

A subject of the invention is also a process for the preparation of a compound of formula (I) as defined above, characterized in that:
the α-amino ester derivative of formula (1)

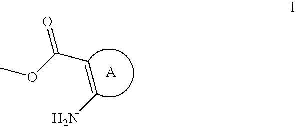

in which A is as defined above, is coupled with the acid chloride of formula R₁COCl in which R₁ is as defined above, in the presence of a tertiary base in an inert organic solvent at ambient temperature for 3 to 24 hours in order to produce compound (2)

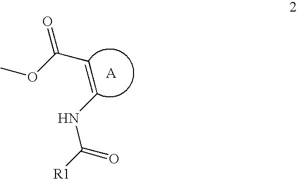

compound (2) thus obtained is saponified in the presence of an inorganic base in a mixture of polar solvents in order to produce the corresponding carboxylic acid (3)

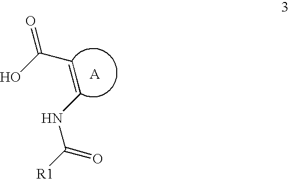

the resultant carboxylic acid (3) is then coupled to a primary amine of formula R₂NH₂ in which R₂ is as defined above in the presence of a coupling agent in an inert organic solvent, in order to produce the corresponding diamide (4).

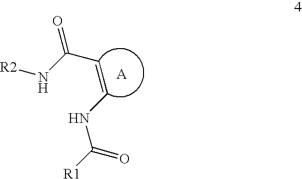

and finally the diamide (4) is cyclized in order to form the pyrimidinone derivative (I) either by treatment with chlorotrimethylsilane (TMSCl) in the presence of a tertiary base in an inert organic solvent at ambient temperature, or by treatment with an inorganic base, in the presence or absence of a transfer agent, in an organic solvent at a temperature of 200 to 250° C.

The compounds of the present invention possess useful pharmacological properties. It is thus that it was discovered that the compounds of the present invention possess a good affinity for certain sub-types of cannabinoid receptors, in particular the CB2 receptors. They are particularly useful for treating the pathological states and the diseases in which one or more cannabinoid receptors are involved.

The compounds of the present invention can thus be used in different therapeutic applications. They can advantageously be used for the treatment and prevention of the pathological states and the diseases associated with the activity of the cannabinoid receptors such as cell proliferation disorders such as cancer, immune disorders, inflammation, pain, osteoporosis, epilepsy, nausea associated with chemotherapy treatments, fibrosis, gastro-intestinal disorders, neurodegenerative diseases including multiple sclerosis and dyskinesia, Parkinson's disease, Huntington's chorea, Alzheimer's disease. They can also be used in order to prevent or cure diseases associated with motor function such as Tourette's syndrome, or in order to provide neuroprotection. The compounds according to the present invention can be administered alone or in combination with other agents linked to treatments of the symptoms or the cause of the disease or pathological state as mentioned above. The experimental part hereafter includes an illustration of the pharmacological properties of the compounds of the invention.

A subject of the present application is also the pharmaceutical compositions containing, as active ingredient, at least one product of formula (I) as defined above, or an addition salt with the pharmaceutically acceptable mineral or organic acids of said product of formula (I), in combination with a pharmaceutically acceptable support.

A subject of the present application is also the use of the compounds (I) according to the present invention, for the preparation of a medicament for the treatment of cell proliferation disorders and preferably for the treatment of cancer.

A subject of the present application is also the use of the compounds (I) according to the present invention, for the preparation of a medicament for the treatment of immune disorders, inflammation, pain, osteoporosis, fibrosis, gastro-intestinal disorders, neurodegenerative diseases including multiple sclerosis and dyskinesia, Parkinson's disease.

The Patent Application US 2005/0176742 describes thiophenepyrimidinone derivatives but these derivatives are presented as 17β-hydroxysteroid dehydrogenase enzyme inhibitors.

A subject of the present application is therefore also the use of the compounds of formula (I′)

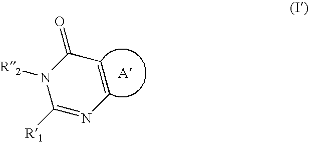

in racemic or enantiomeric form or any combinations of these forms and in which
R′₁ represents a radical corresponding to the anthracene group, a —Y₁—V₁-Z₁ radical or a radical of formula

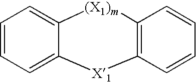

X₁ and X′₁ represent, independently, —CH₂—, —C(O)—, —O—, —S— or —NH—;
m represents 0 or 1;
Y₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
V₁ represents a covalent bond, —O—, —S—, —NH—, —C(O)— or (C₁-C₂)alkyl;
Z₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
R″₂ represents a radical of formula —(CH₂)₂—R′₂;
R′₂ represents a (C₃-C₇)cycloalkyl, bicycloalkyl, heterocycloalkyl, heterobicycloalkyl, cyclohexenyl, aryl or heteroaryl radical, all these radicals being optionally substituted by one or more identical or different substituents chosen from: halo, (C₁-C₆)alkyl, (C₁-C₆)haloalkyl, (C₁-C₆)alkoxy and (C₁-C₆)haloalkoxy;
A′ represents a condensed, unsaturated, aromatic or non-aromatic, mono- or bi-cyclic radical, containing a heteroatom chosen from O and S, and optionally substituted by one or more identical or different radicals, chosen from: halo, nitro, (C₁-C₆)alkyl, (C₁-C₆)haloalkyl, (C₁-C₆)alkoxy, (C₁-C₆)haloalkyl and aryl optionally substituted by one or more substituents chosen from: halo and (C₁-C₆)alkyl; or a pharmaceutically acceptable salt thereof;
for the preparation of a medicament for the treatment of cell proliferation disorders and preferably for the treatment of cancer.

A subject of the present application is also the use of the compounds (1′) as defined above, for the preparation of a medicament for the treatment of immune disorders, inflammation, pain, osteoporosis, fibrosis, gastro-intestinal disorders, neurodegenerative diseases including multiple sclerosis and dyskinesia, Parkinson's disease.

The pharmaceutical composition can be in the form of a solid, for example, powders, granules, tablets, gelatin capsules or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups. Appropriate liquid supports can be, for example, water, organic solvents such as glycerol or the glycols, as well as their mixtures, in varying proportions, in water, added to pharmaceutically acceptable oils or fats. The sterile liquid compositions can be used for intramuscular, intraperitoneal or sub-cutaneous injections and the sterile compositions can also be administered intravenously.

All the technical and scientific terms used in the present text have the meaning known to a person skilled in the art. Moreover, all the patents (or patent applications) as well as the other bibliographical references are incorporated by way of reference.

### Experimental Part

The compounds according to the invention obtained according to the previously described procedures are shown in the table below.

The compounds are characterized by their retention time (rt) and their molecular peak determined by mass spectrometry (MH+).

For the mass spectrometry, a single quadripole mass spectrometer (Micromass, Platform model) equipped with an electrospray source is used with a resolution of 0.8 Da at 50% valley. Calibration is carried out monthly between the masses 80 and 1000 Da using a calibration mixture of sodium iodide of and rubidium iodide in solution in an isopropanol/water mixture (1/1 Vol.).

For the liquid chromatography, a Waters system comprising an in-line degasser, a Waters 600 quaternary pump, a Gilson 233 plate sampling injector and a Waters PAD 996 UV detector is used.

The elution conditions used are as follows:

Eluant: A water+0.02% trifluoroacetic acid; B acetonitrile

| T (min) | A % | B % |
|---|---|---|
| 1 | 95 | 5 |
| 8.5 | 5 | 95 |
| 10.5 | 5 | 95 |
| 10.6 | 95 | 5 |
| 14.9 | 95 | 5 |
| 15.0 | 95 | 5 |

Flow rate: 1 ml/min; Injection: 10 μl; Column: Uptisphere ODS 3 μm 75*4.6 mm i.d.

These examples are presented in order to illustrate the above procedures and should in no event be considered as limiting the scope of the invention.

| Examples | Molecular structures | [M + H]+ | rt (min) |
|---|---|---|---|
| 1 | 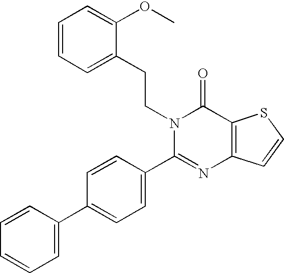 | 439.2 | 10.9 |
| 2 | 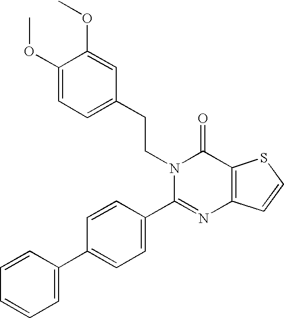 | 469.2 | 10.3 |
| 3 | 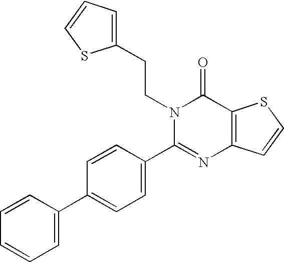 | 415.1 | 10.7 |
| 4 | 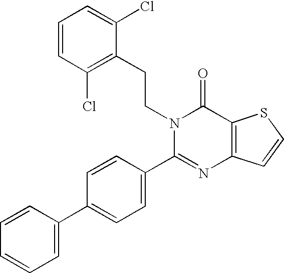 | 477.1 | 11.3 |
| 5 | 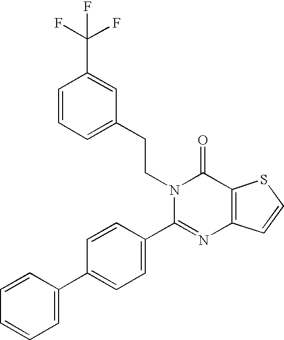 | 477.2 | 11.1 |
| 6 | 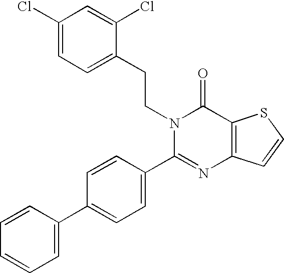 | 477.1 | 11.6 |
| 7 | 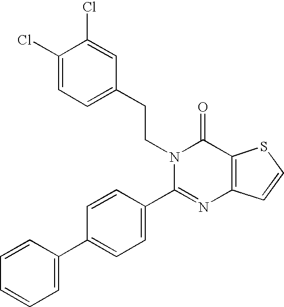 | 477.1 | 11.5 |
| 8 | 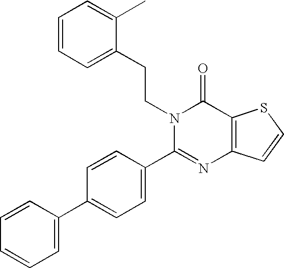 | 423.2 | 11.1 |
| 9 | 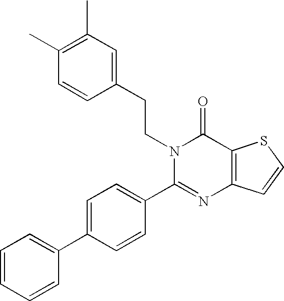 | 437.2 | 11.4 |
| 10 | 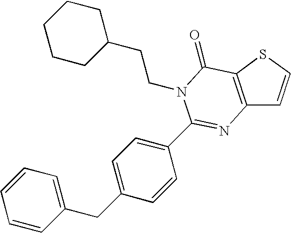 | 429.2 | 13.3 |
| 11 | 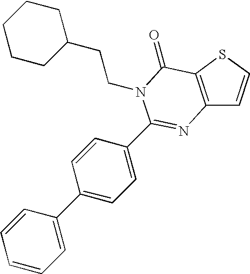 | 415.2 | 13.3 |
| 12 | 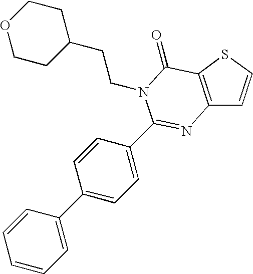 | 417.2 | 11.2 |
| 13 | 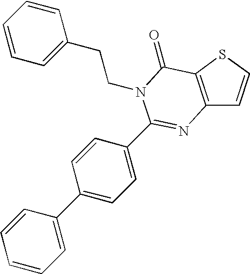 | 409.2 | 12.0 |
| 14 | 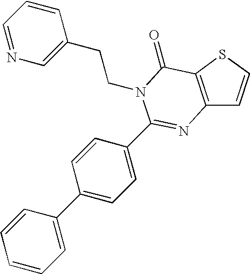 | 410.2 | 8.6 |
| 15 | 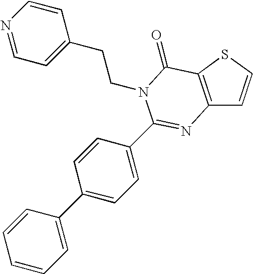 | 410.2 | 8.5 |
| 16 | 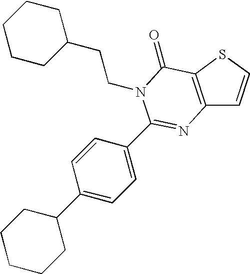 | 421.3 | 13.6 |
| 17 | 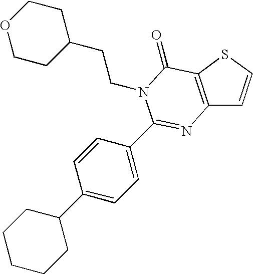 | 423.2 | 11.2 |
| 18 | 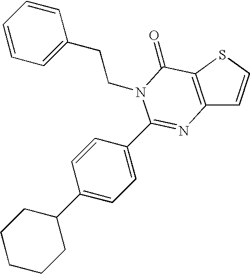 | 415.2 | 11.9 |
| 19 | 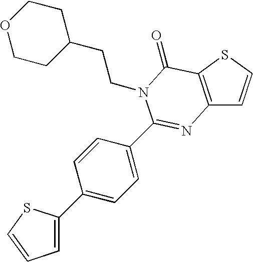 | 423.1 | 9.9 |
| 20 | 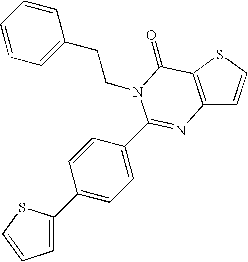 | 415.1 | 10.8 |
| 21 | 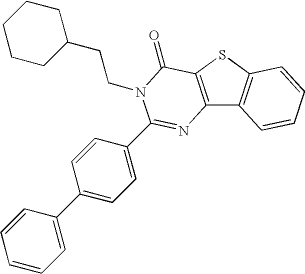 | 465.3 | 13.2 |
| 22 | 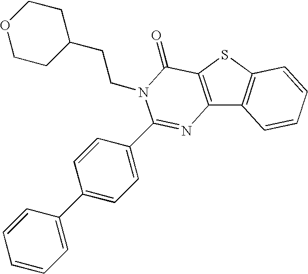 | 467.3 | 11.4 |
| 23 | 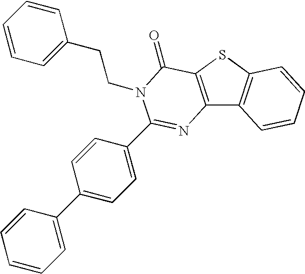 | 459.2 | 11.9 |
| 24 | 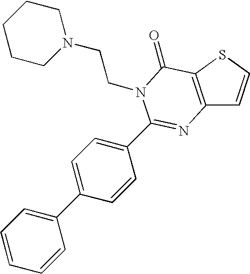 | 416.2 | 8.4 |
| 25 | 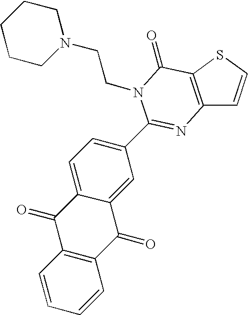 | 470.2 | 8.2 |
| 26 | 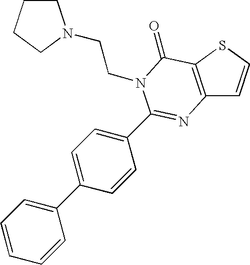 | 402.2 | 8.3 |
| 27 | 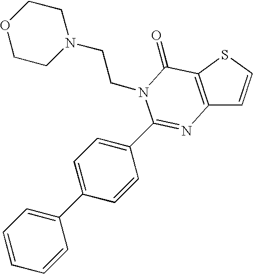 | 418.2 | 8.2 |
| 28 | 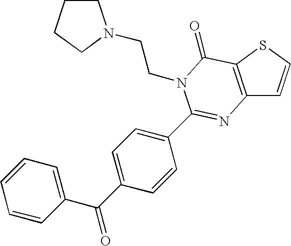 | 430.1 | 8.2 |
| 29 | 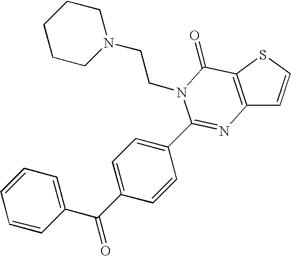 | 444.2 | 8.3 |
| 30 | 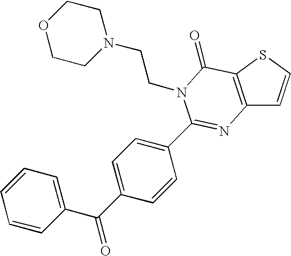 | 446.2 | 8.1 |
| 31 | 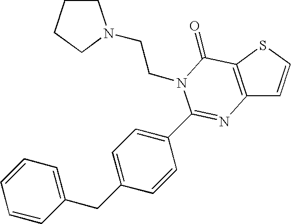 | 416.1 | 8.5 |
| 32 | 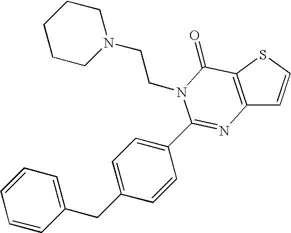 | 430.1 | 8.6 |
| 33 | 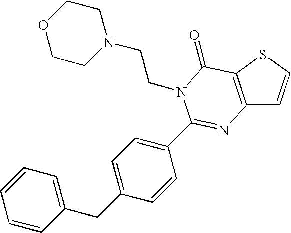 | 432.1 | 8.4 |
| 34 | 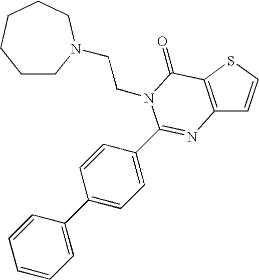 | 430.2 | 8.7 |
| 35 | 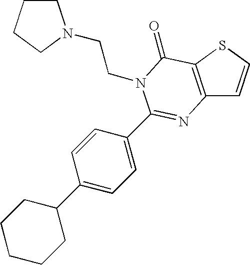 | 408.2 | 8.1 |
| 36 | 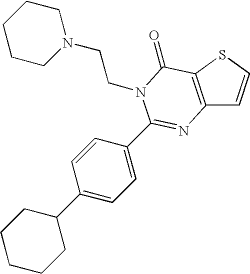 | 422.3 | 8.2 |
| 37 | 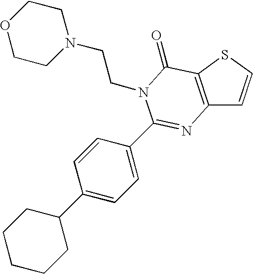 | 424.2 | 8.0 |
| 38 | 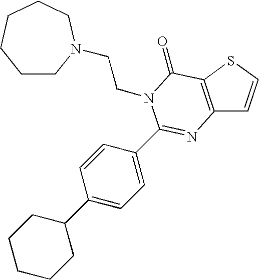 | 436.3 | 8.3 |
| 39 | 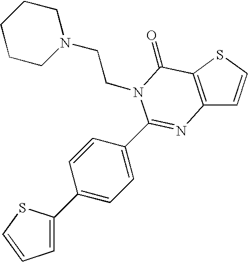 | 422.2 | 7.7 |
| 40 | 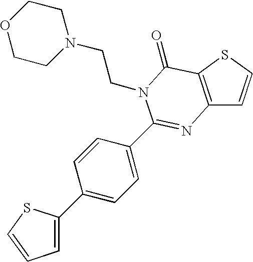 | 424.1 | 7.6 |
| 41 | 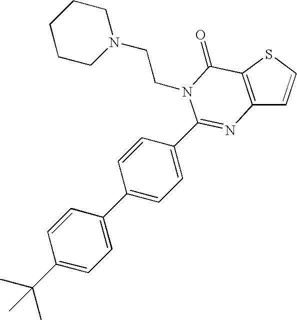 | 472.3 | 8.4 |
| 42 | 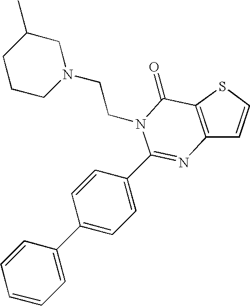 | 430.2 | 8.0 |
| 43 | 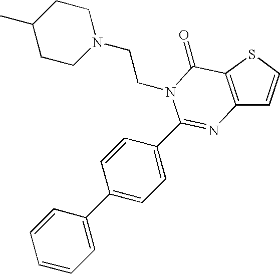 | 430.2 | 8.0 |
| 44 | 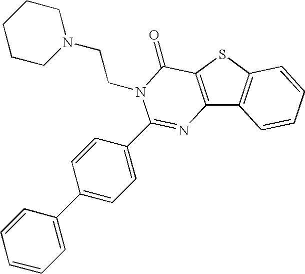 | 466.3 | 8.4 |
| 45 | 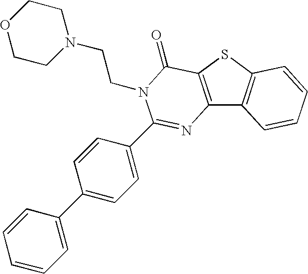 | 468.3 | 8.3 |
| 46 | 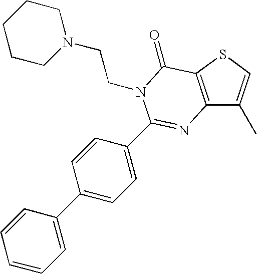 | 430.2 | 8.1 |
| 47 | 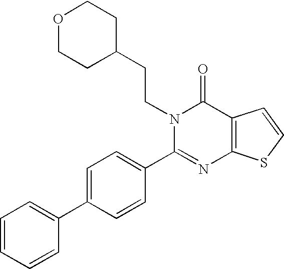 | 417.2 | 11.5 |
| 48 | 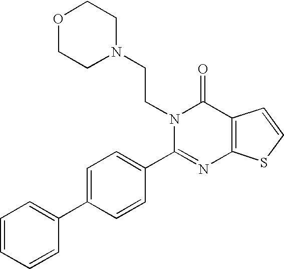 | 418.2 | 8.5 |
| 49 | 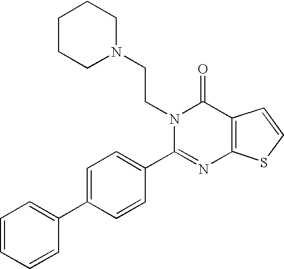 | 416.2 | 8.7 |
| 50 | 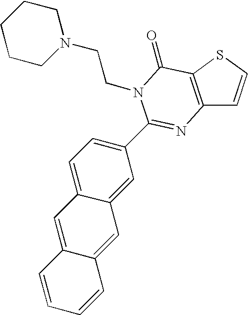 | 440.2 | 8.4 |
| 51 | 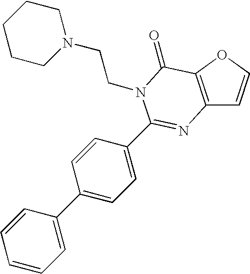 | 400.2 | 8.3 |
| 52 | 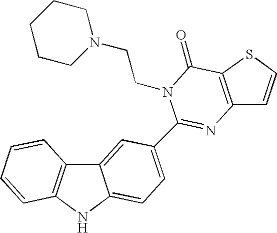 | 429.2 | 7.6 |
| 53 | 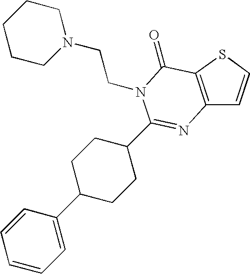 | 422.2 | 9.0 |
| 54 | 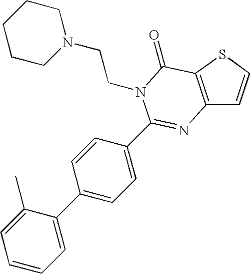 | 430.2 | 8.3 |
| 55 | 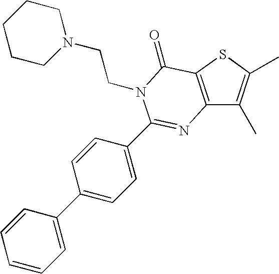 | 444.3 | 8.3 |
| 56 | 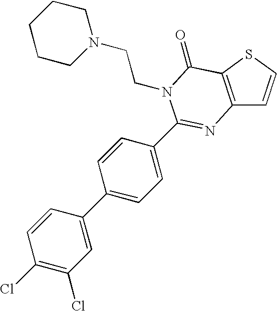 | 484.2 | 8.2 |
| 57 | 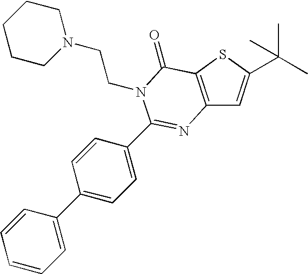 | 472.3 | 8.5 |
| 58 | 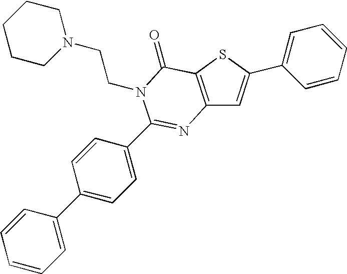 | 492.3 | 8.5 |
| 59 | 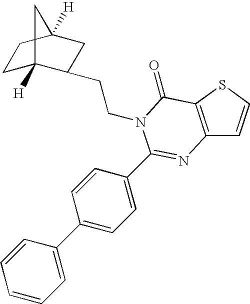 | 427.2 | 11.1 |
| 60 | 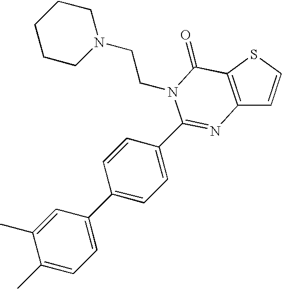 | 444.3 | 8.4 |
| 61 | 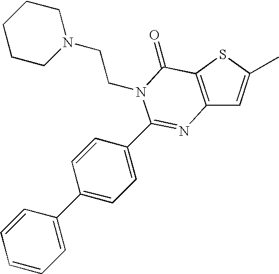 | 430.3 | 7.9 |
| 62 | 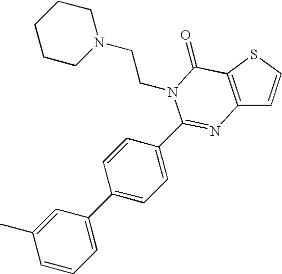 | 430.2 | 8.0 |
| 63 | 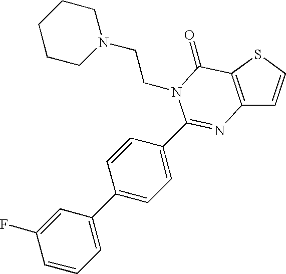 | 434.2 | 7.8 |
| 64 | 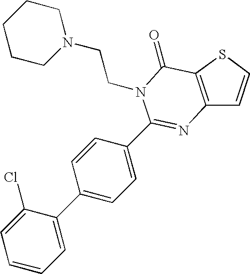 | 450.2 | 7.9 |
| 65 | 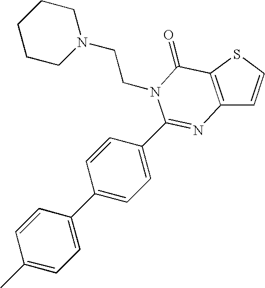 | 430.3 | 8.0 |
| 66 | 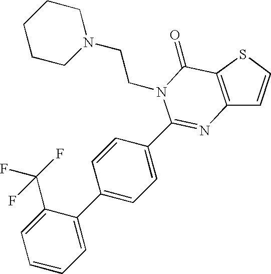 | 484.3 | 8.0 |
| 67 | 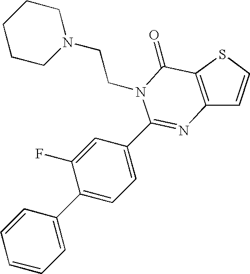 | 434.2 | 8.1 |
| 68 | 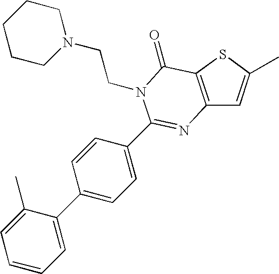 | 444.2 | 8.5 |
| 69 | 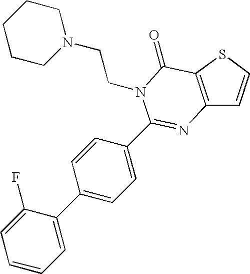 | 434.2 | 8.1 |
| 70 | 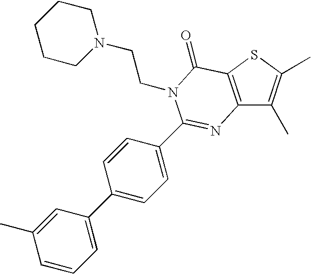 | 458.3 | 9.2 |
| 71 | 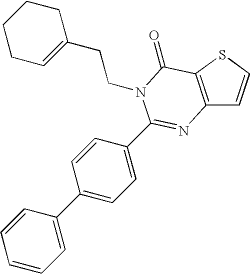 | 413.2 | 10.8 |
| 72 | 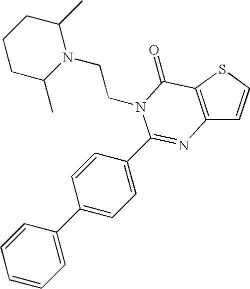 | 444.2 | 8.2 |
| 73 | 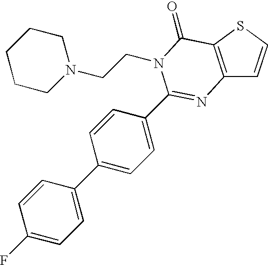 | 434.2 | 8.1 |
| 74 | 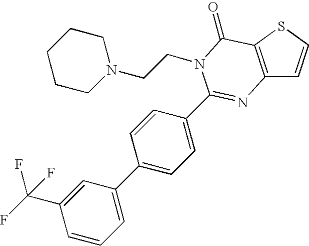 | 484.2 | 8.5 |
| 75 | 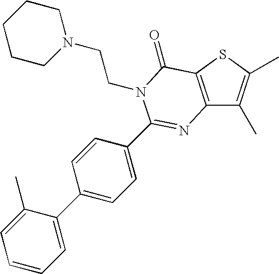 | 458.2 | 9.1 |
| 76 | 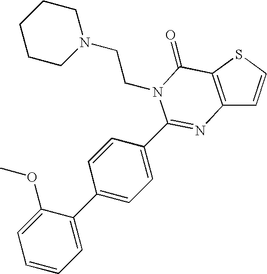 | 446.1 | 8.4 |
| 77 | 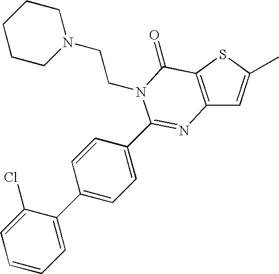 | 464.1 | 8.9 |
| 78 | 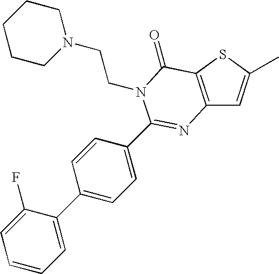 | 448.1 | 8.7 |
| 79 | 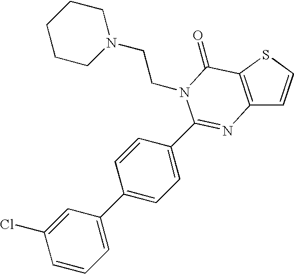 | 450.1 | 8.8 |
| 80 | 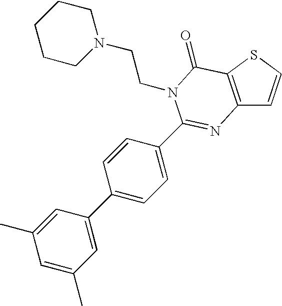 | 444.2 | 9.9 |
| 81 | 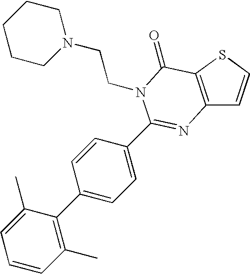 | 444.1 | 8.9 |
| 82 | 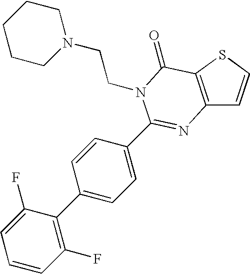 | 452.3 | 8.5 |
| 83 | 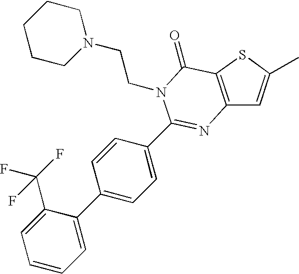 | 498.3 | 9.0 |
| 84 | 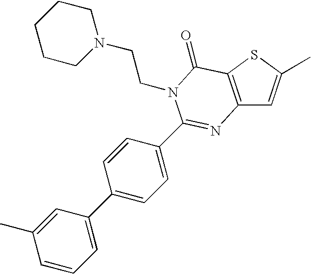 | 444.3 | 8.9 |
| 85 | 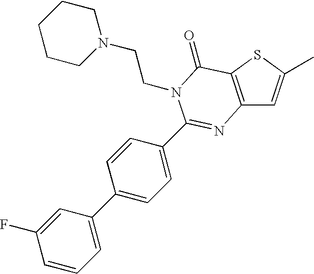 | 448.3 | 8.7 |
| 86 | 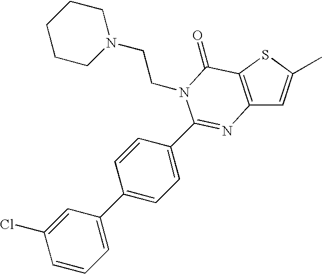 | 464.2 | 9.0 |
| 87 | 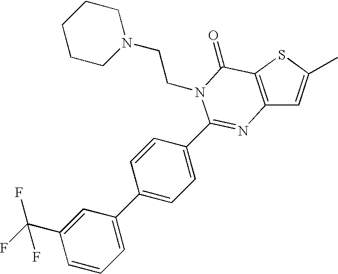 | 498.3 | 9.1 |
| 88 | 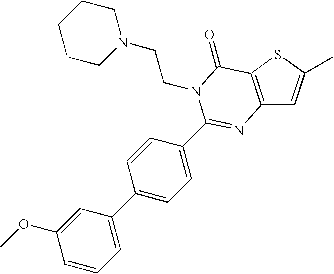 | 460.3 | 8.7 |
| 89 | 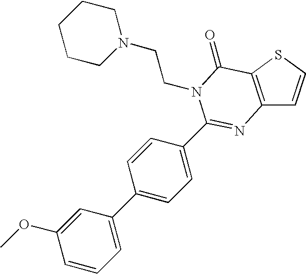 | 446.2 | 8.5 |
| 90 | 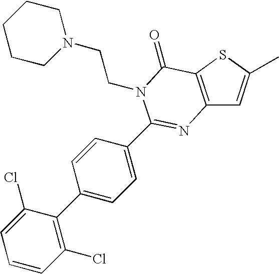 | 498.1 | 9.1 |
| 91 | 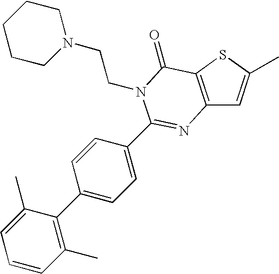 | 458.3 | 9.2 |
| 92 | 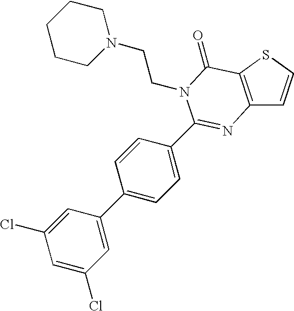 | 484.1 | 9.2 |

### Pharmacological Study

The affinity of the compounds of the present invention for the different sub-types of cannabinoid receptors was measured according to procedures analogous to those described hereafter for the human CB2 receptor.

### Study of the Affinity of the Compounds for the Human CB2 Cannabinoid Receptors:

The affinity of the compounds of the invention for the human CB2 receptors is determined by measuring the inhibition of the binding of [³H]-CP55940 to transfected CHO-K1 cell membrane preparations.

The CHO-K1 cells expressing the human CB2 receptors in stable fashion are cultured in an RPMI 1640 medium containing 10% foetal calf serum, 2 mM of glutamine, 100 U/ml of penicillin, 0.1 mg/ml of streptomycin and 0.5 mg/ml of G418. The cells are collected with 0.5 mM of EDTA and centrifuged at 500 g for 5 minutes at 4° C. The pellet is re-suspended in phosphate buffered saline medium (PBS) and centrifuged at 500 g for 5 minutes at 4° C. The pellet is re-suspended in a Tris 50 mM buffer medium at pH 7.4 and centrifuged at 500 g for 5 minutes at 4° C. The cells are lysed by sonication and centrifuged at 39,000 g for 10 minutes at 4° C. The pellet is re-suspended in the Tris 50 mM buffer medium at pH 7.4 and centrifuged at 50,000 g for 10 minutes at 4° C. The membranes obtained in this last pellet are stored at −80° C.

Measurement of the competitive inhibition of the binding of the [³H]-CP55940 to the CB2 receptors is carried out in duplicate using 96-well polypropylene plates. The cell membranes (10 μg of proteins/well) are incubated with the [³H]-CP55940 (1 nM) for 60 minutes at 25° C. in a Tris-HCl 50 mM buffer medium, pH 7.4, comprising 0.1% bovine serum albumin (BSA), 5 mM of MgCl₂, and 50 μg/ml of bacitracin.

The bound [³H]-CP55940 is separated from the free [³H]-CP55940 by filtration through GF/C glass fibre filter plates (Unifilter, Packard) pre-impregnated with 0.1% polyethylenimine (P.E.I.), using a Filtermate 196 (Packard). The filters are washed with Tris-HCl 50 mM buffer, pH 7.4 at 0-4° C. and the radioactivity present is determined using a counter (Packard Top Count).

The specific binding is obtained by subtracting the non-specific binding (determined in the presence of 0.1 μM of WIN55212-2 from the total binding). The data are analyzed by computer-assisted non-linear regression (MDL). For each test, a Cheng-Prusoff correction is made in order to convert the IC₅₀ to the inhibition constant, Ki.

Thus,

$$Ki = \frac{{IC}_{50}}{1 + [ L ] \text{/} Kd}$$

where [L] is the concentration of the radioligand used in the test and Kd is the radioligand equilibrium dissociation constant.

The inhibition constant value ranges thus measured are presented in the table below.

| Examples | Ki < 5 μM | Ki < 0.5 μM |
|---|---|---|
| 1 | x | |
| 2 | x | |
| 3 | x | |
| 6 | x | x |
| 7 | x | x |
| 8 | x | |
| 9 | x | |
| 11 | x | x |
| 12 | x | x |
| 13 | x | x |
| 14 | x | |
| 15 | x | x |
| 16 | x | x |
| 17 | x | x |
| 18 | x | x |
| 19 | x | |
| 20 | x | x |
| 22 | x | |
| 24 | x | x |
| 25 | x | x |
| 26 | x | x |
| 27 | x | x |
| 29 | x | x |
| 30 | x | x |
| 32 | x | x |
| 34 | x | x |
| 35 | x | x |
| 36 | x | x |
| 37 | x | x |
| 38 | x | x |
| 39 | x | x |
| 40 | x | x |
| 41 | x | |
| 42 | x | x |
| 43 | x | x |
| 44 | x | x |
| 45 | x | |
| 46 | x | x |
| 47 | x | |
| 48 | x | |
| 49 | x | x |
| 50 | x | x |
| 51 | x | |
| 52 | x | |
| 53 | x | x |
| 54 | x | x |
| 55 | x | x |
| 56 | x | x |
| 57 | x | |
| 58 | x | x |
| 59 | x | |
| 60 | x | x |
| 61 | x | x |
| 62 | x | x |
| 63 | x | x |
| 64 | x | x |
| 65 | x | x |
| 66 | x | x |
| 67 | x | x |
| 68 | x | x |
| 69 | x | x |
| 70 | x | x |
| 71 | x | x |
| 72 | x | |
| 73 | x | x |
| 74 | x | x |
| 75 | x | x |
| 76 | x | x |
| 77 | x | x |
| 78 | x | x |
| 79 | x | x |
| 80 | x | x |
| 81 | x | x |
| 82 | x | x |
| 83 | x | x |
| 84 | x | x |
| 85 | x | x |
| 86 | x | x |
| 87 | x | x |
| 88 | x | x |
| 89 | x | x |
| 90 | x | x |
| 91 | x | x |
| 92 | x | x |

The agonistic or antagonistic activity of the CB2 receptors of the compounds of the present invention was determined by measuring the production of cyclic AMP by the CHO-K1 cells transfected by the CB2 receptor.

### Measurement of the Production of Intracellular Cyclic AMP Via the CB2 Receptors:

The CHO-K1 cells expressing the CB2 cannabinoid receptors are cultured in 384-well plates in an RPMI 1640 medium with 10% foetal calf serum and 0.5 mg/ml of G418. The cells are washed twice with 50 μl of RPMI medium comprising 0.2% BSA and 0.5 mM of 3-isobutyl-1-methylxanthine (IBMX).

In order to measure the agonistic effect of a compound, the cells are incubated for 5 minutes at 37° C. in the presence of 0.5 mM of IBMX, then the stimulation of the production of cyclic AMP is obtained by adding 5 μM of Forskolin then the inhibition is measured by the addition of compound at concentrations comprised between 1 μM and 10 μM in duplicate for 20 minutes at 37° C. The antagonistic effect of a compound is measured by inhibiting the inhibition of the production of cyclic AMP induced by WIN55212-2 in the presence of 5 μM of Forskolin, at concentrations comprised between 1 pM and 10 μM, in the presence of the compound to be tested, at concentrations comprised between 1 nM and 10 μM, in duplicate for 20 minutes at 37° C.

The reaction medium is eliminated and 80 μl of lysis buffer is added. The level of intracellular cyclic AMP is measured by a competition test with fluorescent cyclic AMP (CatchPoint, Molecular Devices).

1. Compounds of general formula (I) 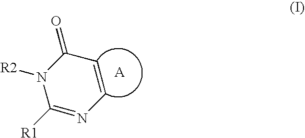
in racemic or enantiomeric form or any combinations of these forms and in which:
R₁ represents a radical corresponding to the anthracene group, a —Y₁—V₁-Z₁ radical or a radical of formula 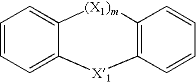
X₁ and X′₁ represent, independently, —CH₂—, —C(O)—, —O—, —S— or —NH—;
m represents 0 or 1;
Y₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
V₁ represents a covalent bond, —O—, —S—, —NH—, —C(O)— or (C₁-C₂)alkyl;
Z₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
R₂ represents a radical of formula —(CH₂)₂—R′₂;
R′₂ represents a (C₃-C₇)cycloalkyl, bicycloalkyl, heterocycloalkyl, heterobicycloalkyl, cyclohexenyl, aryl or heteroaryl radical, all these radicals being optionally substituted by one or more identical or different substituents chosen from: halo, (C₁-C₆)alkyl, (C₁-C₆)haloalkyl, (C₁-C₆)alkoxy and (C₁-C₆)haloalkoxy;
A represents a condensed, unsaturated, aromatic or non-aromatic, mono- or bi-cyclic radical, comprising a heteroatom chosen from O and S, and optionally substituted by one or more identical or different radicals, chosen from: halo, nitro, (C₁-C₆)alkyl, (C₁-C₆)haloalkyl, (C₁-C₆)alkoxy and (C₁-C₆)haloalkyl and aryl optionally substituted by one or more substituents chosen from: halo and (C₁-C₆)alkyl;
or pharmaceutically acceptable salt thereof;
excluding the compounds of formula (Ic) 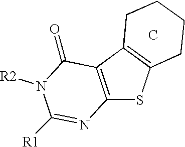
in which the C ring is an unsaturated carbon-containing ring including at the most 3 double bonds and optionally substituted

2. Compounds of general formula (I) as defined in claim 1, wherein:
R₁ represents a radical corresponding to the anthracene group, a radical of formula —Y₁—V₁-Z₁ or of formula 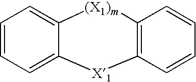
X₁ and X′₁ represent, independently, —CH₂—, —C(O)— or —NH—;
m represents 0 or 1;
Y₁ represents a (C₃-C₇)cycloalkyl, or aryl radical optionally substituted by one or more identical or different halo substituents;
V₁ represents a covalent bond, —O—, —C(O)— or —CH₂—;
Z₁ represents a (C₃-C₇)cycloalkyl, or aryl or heteroaryl radical, all these radicals being optionally substituted by one or more identical or different substituents chosen from: halo, (C₁-C₆)alkyl, (C₁-C₆)haloalkyl and (C₁-C₆)alkoxy.

3. The compounds of claim 1, wherein:
Y₁ represents a cyclohexyl or phenyl radical optionally substituted by one or more identical or different halo substituents;
Z₁ represents a cyclohexyl, phenyl or thienyl radical, all these radicals being optionally substituted by one or more identical or different substituents chosen from: halo, (C₁-C₆)alkyl, (C₁-C₆)haloalkyl and (C₁-C₆)alkoxy.

4. The compounds of claim 1, wherein:
R′₂ represents a (C₃-C₇)cycloalkyl, bicycloalkyl, heterocycloalkyl, cyclohexenyl, aryl or heteroaryl radical, all these radicals being optionally substituted by one or more identical or different substituents chosen from: halo, (C₁-C₆)alkyl, (C₁-C₆)haloalkyl, and (C₁-C₆)alkoxy.

5. The compounds of claim 1, wherein the (C₃-C₇)cycloalkyl, bicycloalkyl, heterocycloalkyl, aryl or heteroaryl radical represented by R′₂ is chosen from: cyclopentyl, cyclohexyl, cycloheptyl, bicyclo[2,2,1]heptyl, phenyl, pyrrolidinyl, piperidinyl, azepanyl, morpholinyl, tetrahydropyranyl, pyridinyl and thienyl.

6. The compounds of claim 1, wherein A represents an aromatic radical optionally substituted by one or more identical or different substituents chosen from (C₁-C₆)alkyl and aryl.

7. The compounds of claim 1, wherein A represents the thienyl, furyl or benzothienyl radical, said radicals being optionally substituted by one or more identical or different substituents chosen from (C₁-C₆)alkyl and phenyl.

8. The compounds of claim 1, wherein:
R₁ represents a radical of formula —Y₁—V₁-Z₁;
Y₁ represents a cyclohexyl or phenyl radical;
V₁ represents a covalent bond;
Z₁ represents a cyclohexyl or phenyl radical optionally substituted by one or more identical or different halo, (C₁-C₆)alkyl, or (C₁-C₆)haloalkyl substituents.

9. The compounds of claim 1, wherein R′₂ represents the piperidinyl, azepanyl, morpholinyl, tetrahydropyranyl, cyclohexyl or cyclohexenyl radical.

10. The compounds of claim 6, wherein A represents a monocyclic radical.

11. The compounds of claim 1, wherein A represents the thienyl radical optionally substituted by one or more identical or different (C₁-C₆)alkyl substituents.

12. The compounds of claim 6, wherein A represents a bicyclic radical.

13. A compound of claim 12, wherein A forms with the pyrimidinone ring, the following compound

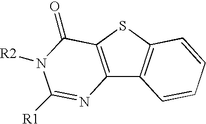

14. A method for producing the compounds of claim 1, comprising the steps of:
1. coupling the α-amino ester derivative of formula (1) 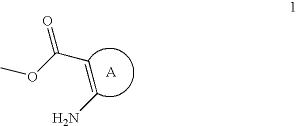
in which A is as defined in claim 1, to the acid chloride of formula R₁COCl in which R₁ is as defined in claim 1, in the presence of a tertiary base in an inert organic solvent at ambient temperature for 3 to 24 hours in order to produce compound (2) 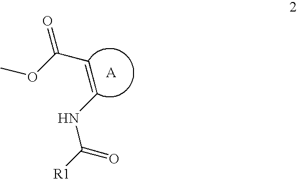
2. saponifying compound (2) thus obtained in the presence of an inorganic base in a mixture of polar solvents in order to produce the corresponding carboxylic acid (3) 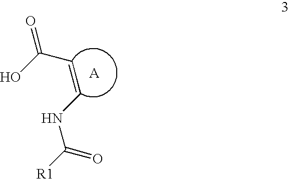
3. coupling the resultant carboxylic acid (3) to a primary amine of formula R₂NH₂ in which R₂ is as defined in claim 1, in the presence of a coupling agent in an inert organic solvent, in order to produce the corresponding diamide (4) 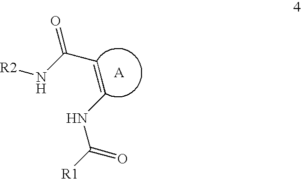
4. cyclizing the diamide (4) in order to form the pyrimidinone derivative (I) either by treatment with chlorotrimethylsilane (TMSCl) in the presence of a tertiary base in an inert organic solvent at ambient temperature, or by treatment with an inorganic base, in the presence or absence of a transfer agent, in an organic solvent at a temperature of 200 to 250° C.

15. Pharmaceutical compositions comprising, as an active ingredient, at least one of the compounds of claim 1, or an addition salt with pharmaceutically acceptable mineral or organic acids of said compound of claim 1, in combination with a pharmaceutically acceptable support.

16. A method for the treatment of cell proliferation disorders, comprising administering to a patient in need thereof a therapeutically effective amount of a compound of claim 1.

17. A method for the treatment of immune disorders, inflammation, pain, osteoporosis, fibrosis, gastro-intestinal disorders, neurodegenerative diseases including multiple sclerosis and dyskinesia, or Parkinson's disease comprising administering to a patient in need thereof a therapeutically effective amount of a compound of claim 1.

18. A method for the treatment of cell proliferation disorders, comprising administering to a patient in need thereof a therapeutically effective amount of a compound of formula (I′) 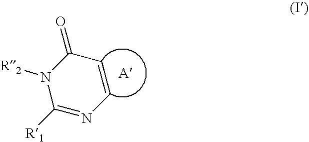
in racemic or enantiomeric form or any combinations of these forms and in which:
R′₁ represents a radical corresponding to the anthracene group, a —Y₁—V₁-Z₁ radical or a radical of formula 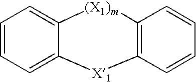
X₁ and X′₁ represent, independently, —CH₂—, —C(O)—, —O—, —S— or —NH—;
m represents 0 or 1;
Y₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
V₁ represents a covalent bond, —O—, —S—, —NH—, —C(O)— or (C₁-C₂)alkyl;
Z₁ represents a (C₃-C₇)cycloalkyl, heterocycloalkyl, aryl or heteroaryl radical, all these radicals being optionally substituted by one or more identical or different substituents chosen from: halo, nitro, (C₁-C₆)alkyl, (C₁-C₆)haloalkyl, (C₁-C₆)alkoxy, (C₁-C₆)haloalkoxy and (C₁-C₆)alkyl-C(O)—;
R″₂ represents a radical of formula —(CH₂)₂—R′₂;
R′₂ represents a (C₃-C₇)cycloalkyl, bicycloalkyl, heterocycloalkyl, heterobicycloalkyl, cyclohexenyl, aryl or heteroaryl radical, all these radicals being optionally substituted by one or more identical or different substituents chosen from: halo, (C₁-C₆)alkyl, (C₁-C₆)haloalkyl, (C₁-C₆)alkoxy and (C₁-C₆)haloalkoxy;
A′ represents a condensed, unsaturated, aromatic or non-aromatic, mono- or bi-cyclic radical, containing a heteroatom chosen from O and S, and optionally substituted by one or more identical or different radicals, chosen from: halo, nitro, (C₁-C₆)alkyl, (C₁-C₆)haloalkyl, (C₁-C₆)alkoxy, (C₁-C₆)haloalkyl and aryl optionally substituted by one or more substituents chosen from: halo and (C₁-C₆)alkyl;
or a pharmaceutically acceptable salt thereof.

19. A method for the treatment of immune disorders, inflammation, pain, osteoporosis, fibrosis, gastro-intestinal disorders, neurodegenerative diseases, or Parkinson's disease comprising administering to a patient a therapeutically effective amount of a compound of claim 18.

20. A method for the treatment of cancer, comprising administering to a patient in need thereof a therapeutically effective amount of a compound of claim 1.

21. The method of claim 18, wherein the cell proliferation disorder is cancer.

22. The method of claim 19, wherein the neurodegenerative disease is multiple sclerosis or dyskinesia.
